(19) United States
(12) Patent Application Publication
(10) Pub. No.: US 2024/0389861 A1
(43) Pub. Date: Nov. 28, 2024

(54) WIDE-FIELD PHOTOACOUSTIC (PA) IMAGING SYSTEM

(71) Applicant: Worcester Polytechnic Institute, Worcester, MA (US)

(72) Inventors: Haichong Zhang, Shrewsbury, MA (US); Shang Gao, Worcester, MA (US); Xihan Ma, Worcester, MA (US)

(21) Appl. No.: 18/674,020

(22) Filed: May 24, 2024

Related U.S. Application Data
(60) Provisional application No. 63/468,892, filed on May 25, 2023.

(51) Int. Cl. A61B 5/00; A61B 34/10; A61B 34/20; A61B 34/30; A61B 90/00; A61M 25/01

(52) U.S. Cl. CPC A61B 5/0095; A61B 5/6852; A61B 34/30; A61B 2034/107; A61B 2034/2055; A61B 2034/301; A61B 2090/3937; A61M 2025/0166

(57) ABSTRACT

A photoacoustic (PA) sensor device has a localization feature that defines a position of the sensor or scan information with a coordinate frame defining relative positions a scanned specimen and sensor information of other imaging planes of the scanned specimen. The coordinate frame is defined by positional information from localization markers at a known offset from the sensor, or from position signals from a robotic actuator driving the sensor. A processor coalesces the sensor information and positional information to reconstruct a 3-dimensional rendered structure by stitching together the sensor information to form a continuous rendering. Stitching may include adjacent imaging planes at an angular offset due to a varied pose of the sensor, and/or adjacent images over a lateral area too large for a single scan or imaging plane to capture.

### RELATED APPLICATIONS

This patent application claims the benefit under 35 U.S.C. § 119(e) of U.S. Provisional Patent App. No. 63/468,892, filed May 25, 2023, entitled “WIDE-FIELD PHOTOACOUSTIC (PA) IMAGING SYSTEM,” incorporated herein by reference in entirety.

### STATEMENT OF FEDERALLY SPONSORED RESEARCH AND DEVELOPMENT

This invention was made with government support under grant No. DP5 OD028162, awarded by the National Institute of Health. The Government has certain rights in the invention.

### BACKGROUND

Ultrasonic sensors use sound waves, typically above the 20 kHz range, to detect objects in proximity. The ultrasound medium avoids harmful emissions such as x-rays and is more compact than Magnetic Resonance Imaging (MRI), hence can be portable. Other common uses include the automotive space, where ultrasonic sensors are prevalent for ADAS (Advanced Driver-Assistant Systems) applications, specifically for parking assist where perimeter-located sensors are used to detect obstacles when parking a vehicle. In the industrial space, ultrasonic sensors are used in robotics and other applications that require reliable presence, proximity, or position sensing.

Ultrasonic sensors can measure distance and detect the presence of an object without making physical contact, by producing and monitoring an ultrasonic echo. Detection in variances in object density can also be used for medical imaging to depict different tissue regions based on varied density. Depending on the sensor and object properties, an effective range in air is between a few centimeters up to several meters. The ultrasonic sensor (or transducer) generates and emits ultrasonic pulses that are reflected back towards the sensor by an object that is within the sensory field and range.

Photoacoustic (PA) imaging is an emerging biomedical imaging modality based on laser-generated ultrasound (US) providing high-resolution, real-time functional information of anatomy. PA imaging has been well-investigated in various applications including vascular mapping, blood oxygenation mapping, tumor detection, ablation monitoring as well as catheter tracking. The imaging modality has been demonstrated for guiding procedures (cardiac ablation, prostatectomy, hysterectomy, etc.) intraoperatively.

### SUMMARY

A photoacoustic (PA) sensor device has a localization feature that defines a position of the sensor or scan information with a coordinate frame defining relative positions a scanned specimen and sensor information of other imaging planes of the scanned specimen. The coordinate frame is defined by positional information from localization markers at a known offset from the sensor, or from position signals from a robotic actuator driving the sensor. A processor coalesces the sensor information and positional information to reconstruct a 3-dimensional rendered structure by stitching together the sensor information to form a continuous rendering. Stitching may include adjacent imaging planes at an angular offset due to a varied pose of the sensor, and/or adjacent images over a lateral area too large for a single scan or imaging plane to capture.

Configurations herein are based, in part, on the observation that PA imaging mediums are often used for medical and laboratory research involving human or animal tissue where other scanning mediums (CT, MRI, X-Ray) are either harmful and/or insufficiently portable. Unfortunately, conventional approaches to PA imaging suffer from the shortcoming that imaging frequently requires a series of scan information taken from varied positions over or adjacent to a scanned specimen (subject). Accordingly, configurations herein substantially overcome the shortcomings of conventional approaches by combining localization information from a common plane of reference to a set of sensor data defined by imaging planes. The orientation between successive or adjacent imaging planes can vary from a parallel relation to an angular offset depending on a speed and pose of the sensor. The result is a true 3 dimensional rendering or representation based on a coalescing or stitching successively gathered imaging plane data.

Various photoacoustic (PA) imaging devices have been reported by conventional approaches, however, these devices typically rely on an optical source that is aligned with an ultrasound (US) array for data acquisition. The majority of existing systems use a 1D-aligned ultrasound array, resulting in a 2D PA image. To acquire a volumetric PA image, the PA probe must be mechanically actuated. For greater clinical flexibility, portable PA systems are emerging that allow physicians to hold the probe and perform the scan. While a larger area of biological tissue can be covered using the hand-held probe, the collected PA images still lack localization information, making it problematic to globally visualize and quantify the interested features in locally acquired images.

### BRIEF DESCRIPTION OF THE DRAWINGS

The foregoing and other objects, features and advantages of the invention will be apparent from the following description of particular embodiments of the invention, as illustrated in the accompanying drawings in which like reference characters refer to the same parts throughout the different views. The drawings are not necessarily to scale, emphasis instead being placed upon illustrating the principles of the invention.

FIG. 1 is a context diagram of a photoacoustic (PA) environment suitable for use with configurations herein;

FIGS. 2A-2C show a configuration of a laparoscopic sensor in the environment of FIG. 1;

FIG. 3 is a configuration of a robotically actuated sensor approach;

FIGS. 4A-4C show a scan specimen and results based on the configuration of FIG. 3;

FIG. 5 show a laparoscopic use case based on the configuration of FIGS. 2A-2C; and

FIG. 6 shows a graphical depiction of the set of scan data defined by a plurality of gathered imaging planes in the approach of FIG. 5.

### DETAILED DESCRIPTION

Configurations herein depict example configuration of a PA device for capturing image information in the imaging plane defined by an overlap of the sensing region of the US array and the irradiation region for each illumination source.

PA imaging receives image information in an acoustic signal similar to an US sensor, but the PA medium induces or generates the acoustic signal differently. US sensing emits and receives the acoustic signal from the same transducer, which both emits and receives the US signal. The PA approach induces an acoustic return signal by an irradiating light signal, rather than an acoustic/sound signal.

In photoacoustic (PA) imaging, ultrasound waves are produced by irradiating the tissue with modulated electromagnetic radiation, usually pulsed on a nanosecond timescale. In the case of optical excitation, absorption by specific tissues such as hemoglobin, melanin, or water followed by rapid conversion to heat produces a small temperature rise. This rise of temperature (i.e., thermal expansion) leads to an initial pressure increase, which then subsequently relaxes, resulting in the emission of broadband low-amplitude acoustic waves. The acoustic waves propagate through the tissue to the surface, where they are detected by the ultrasound receiver. By measuring the time of arrival of the acoustic waves and knowing the speed of sound in tissue, a PA image can be reconstructed in the same way that a pulse-echo ultrasound image is formed. The acoustic pressures in PA are several orders of magnitude smaller than that in ultrasound.

In an US medium, an image represents the acoustic impedance mismatch between different tissues. A PA image, however, is absorption-based. It represents the initial pressure distribution produced by the deposition of the optical energy, which depends on the optical absorption and scattering properties of the tissue. PA imaging can provide greater tissue differentiation and specificity than ultrasound because the difference in optical absorption of tissues can be much larger than the difference in acoustic impedance. PA imaging thus provides the ability to distinguish structures having a higher optical absorption than surrounding tissue; some examples are blood vessels and nerves.

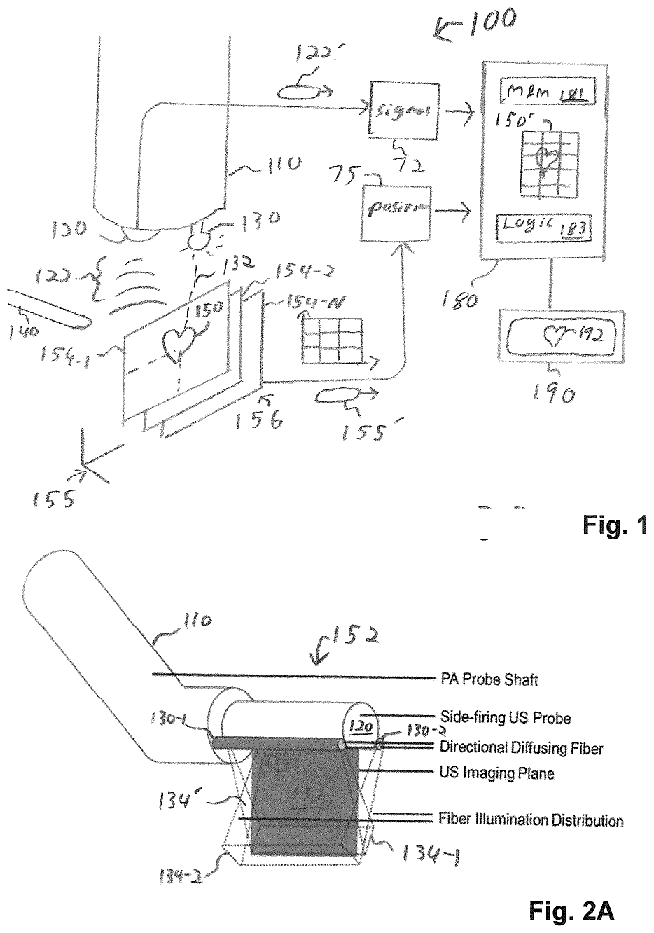

FIG. 1 is a context diagram of a photoacoustic (PA) environment suitable for use with configurations herein. Referring to FIG. 1, in the PA environment 100, a probe 110 and a sensor 120 are deployed for receiving PA signals 122 in response to irradiation 132 (light). The sensing medium for both US and PA is a transducer for receiving acoustic pressure waves—the difference is in the stimuli (sound or light) that induces the acoustic pressure waves.

In the environment of FIG. 1, a specimen 150 resides within range of the probe 110. The sensor 120 receives the PA signals indicative of a 2 dimensional imaging plane 154-1 . . . 154-N(154 generally). The imaging plane 154 resides in a coordinate frame 155. A sensor message 122′ includes sensor signal information 72, and a position message 155′ includes position information 75. A processor 180 combines or coalesces the sensor information 72 and position information 75 to render a localized image 192 on a rendering device 190 such as a surgical display or analysis platform.

In a general usage, the medical imaging device as disclosed herein employs a photoacoustic sensor 120 responsive to photoacoustic signals 132 generated based on an imaging plane 154 designating a planar segment or “slice” through a specimen 150 or subject. A memory 181 is operable to store position information 75 of a coordinate frame 155 defined over an imaged region 156, and an image sequence includes a plurality of imaging planes 154-1 . . . 154-N, such that each imaging plane 154 of the plurality of imaging planes 154-N defined by the photoacoustic signals 122 represents a two dimensional scan of the imaged region 156 received by the photoacoustic sensor 120. A laparoscopic probe or tool 140 may also be present.

A processor 180 includes imaging logic 183 for coalescing each of the imaging planes 154 of the imaged region 156 with a relative location in the coordinate frame 155. A rendering device 190 renders a representation 192 of the imaged region 156 by reconstructing the representation based on the plurality of imaging planes 154 and the corresponding relative location. The coordinate frame 155 for generating the scanned image is indicative of, for each of the imaging planes, a coordinate-based position in the coordinate frame, thus allowing accurate 3 dimensional reconstruction 150′ by locating each imaging plane 154 according to the position information placing it in the coordinate frame 155.

Various approaches to coalescing the sensor information 72 and position information 75 may be pursued, presented further below.

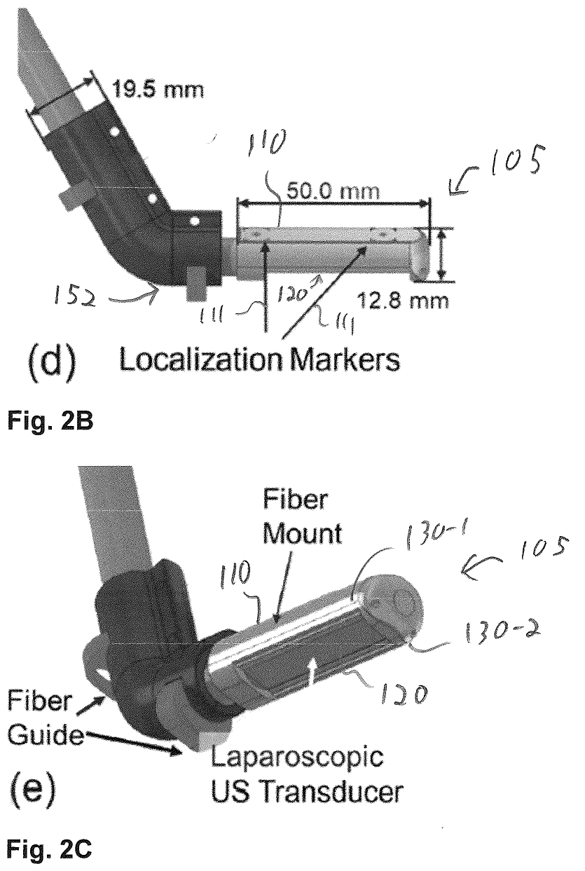

FIGS. 2A-2C show a configuration of a laparoscopic sensor in the environment of FIG. 1. FIG. 2A is a perspective view of a PA probe in the environment of FIG. 1. The use of diffusing fibers, defined by individual optical fibers irradiating a linear region, delivers illumination for PA excitation in a miniature form factor adapted for insertion to the surgical site. Diffusing fibers can illuminate a wider region than the same number of angled-tip side illumination fibers. Thus, an integrated PA imaging device with a miniaturized diameter and simplified alignment mechanism can provide intraoperative guidance with a flexible imaging angle, as shown in FIG. 2A. The imaging probe can be a stand-alone or integrated to be the part of robotics arms of a surgical system.

Referring to FIGS. 1 and 2A-2C, a laparoscopic configuration of the photoacoustic imaging device is shown. FIG. 2A is a schematic of basic function. The PA probe 110 takes the form of a shaft, typically flexible, housing optic fiber(s) and control wires that connect to the ultrasonic sensor 120, which extends longitudinally for forming a linear sensing area 131, defining the sensing region 132 from a rectangular region extending from the sensing area 131 which may be formed from a liner array of sensors and perpendicular from an axis of the shaft. One or more (typically 2) illumination sources 130-1 . . . 130-2 (130 generally), flank the sensor 120, slightly below a diameter of the shaft, extending longitudinally for irradiating the sensing region aligned with the linear sensing area. Each illumination source 130-N irradiates an irradiation region 134-1 . . . 134-2 (134 generally).

Referring to FIGS. 2B and 2C, a pair of localization markers 111 provide a visual reference for alignment with the coordinate frame 155. The imaging plane 154 is defined by the intersection of the sensing region 132 and all of the overlapping irradiation regions 134-N formed from the respective illumination sources 130. 'In the example configuration, the illumination sources 130 are defined by diffusion fibers formed at distal ends of optical fibers 142 terminating at the distal end 105.

FIGS. 2A-2C illustrate a side firing PA probe having a plurality of illumination sources 130-N, such that each illumination source of the plurality of illumination sources extends in parallel adjacent the liner sensing area 131 for defining an intersecting irradiation region 134′ emanating from the parallel illumination sources 130. The natural “fan out” of the illumination sources forms the intersection. The sensing region 132 of the US sensor also meets this intersecting irradiation region 134′. The ultrasonic sensor 120 is responsive to an imaging plane 154, defined by a region parallel to the linear sensing area 131 and extending perpendicular to the longitudinal extension of the ultrasound sensor 120, and within the intersecting irradiation region 134′ resulting from irradiation of light from each of the respective illumination sources 130.

The collective assembly forms a photoacoustic bundle 152, defined by the plurality of illumination sources 130 formed from diffusion fibers flanking the ultrasonic sensor 120, such that each of the diffusion fibers terminates in a respective illumination source 130-N, and thus the imaging plane 154 is defined by an intersection of the irradiation regions 134 extending from each of the illumination sources 130 and the sensing region 132 of the ultrasonic sensor 120. In a particular configuration, the illumination sources may be shaved or ground optic fibers with cladding removal, as disclosed in copending U.S. patent application Ser. No. 18/667,740, filed on May 17, 2024, entitled “SPECTROSCOPIC PHOTOACOUSTIC IMAGING PROBE,” by the assignee of the present application and incorporated herein by reference in entirety.

As a practical matter, the probe 110 is elongated for extension into a surgical region, where the imaging plane 154 passes through the surgical region for imaging thereof. In various contexts, the probe 110 may have a diameter of around 4 mm, suited for insertion into a borehole of around 12-14 mm or a laparoscopic incision. The probe 110 and bundle 152 may also be deployed with other instruments, such as an ablation antenna, other probe or surgical/laparoscopic apparatus. By disposing in or adjacent the surgical region, the ultrasonic sensor 120 is responsive to changes in tissue density of the surgical region resulting from at least one of a vasculature, a tumor or necrosis, for example.

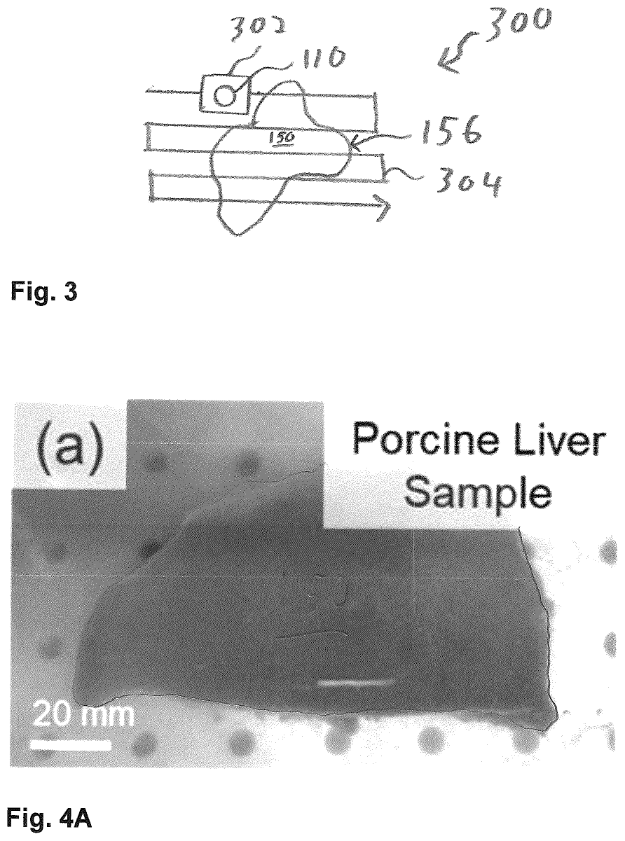

FIG. 3 is a configuration of a robotically actuated sensor approach. Referring to FIGS. 1 and 3, a sensing environment 300 includes a robotic actuator 302, such that the photoacoustic sensor 120 is attached to the robotic actuator 302. The robotic actuator 302 is adapted traverse a predetermined path 304 relative to the imaged region 156 containing the specimen 150. The logic 183 computes the coordinate based position of the respective imaging plane 154 based on a position on the predetermined path 304 when the photoacoustic signals 122 corresponding to the imaging plane 154 are received by the photoacoustic sensor 120. In the example shown, the robotic actuator 302 follows a series of parallel swaths along a plane adjacent to the imaged region 156, i.e. slightly above. Since the predetermined path orients the photoacoustic sensor 120 at a known rate and position, the reference to the coordinate frame 155 can be easily determined and correlated to the signals 122 gathered at the computed position.

The approach of FIG. 3 is useful for a stationary specimen 150. In a particular example, the specimen is a liver under consideration for transplant. Liver ischemia and reperfusion injury (IRI) is a vascular complication that can lead to hepatic functional impairment. IRI is relevant in liver transplantation and during liver surgery performed with intermittent vascular inflow occlusion. Detecting intraoperative liver oxygenation impairment and ischemia could help prevent IRI, which is challenging partially due to the multiple hepatic vascular inflows. The disclosed robot—assisted wide-area photoacoustic (PA) imaging system monitors the liver oxygenation level intraoperatively. PA imaging is suitable for oxygenation detection, as is known in the relevant field. Applying the approach of FIG. 3, a robotic arm actuates the 2D PA probe 110 and scan across the liver surface. An accurate volumetric tomography can be reconstructed via registering 2D images to the recorded robot trajectory. An ex vivo porcine liver sample was scanned using the example of FIG. 3.

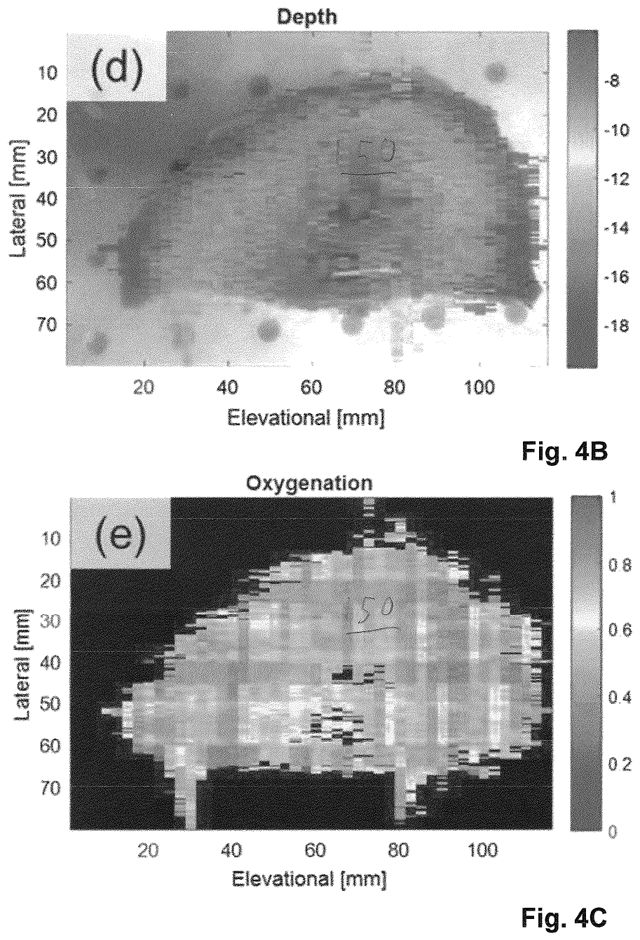

FIGS. 4A-4C show a scan specimen and results based on the configuration of FIG. 3. By registering the 2D images to the recorded robot's trajectory, an accurate volumetric tomography of the liver can be reconstructed. The disclosed example implements a move-stop-scan-move behavior to image the sample. Specifically, the robotic actuator 302 will first travel along a scan path, stop after a small, fixed interval, scan the tissue at the point of stopping, then resume motion along the scan path.

To comprehensively cover the surface of the imaging sample 150, the robotic actuator 302 holds the PA tomographic probe 110 and performs a constant velocity raster-pattern scanning path 304. The probe 110 stops at a fixed interval while moving along the scanning path and acquires multi-wavelength PA imaging before resuming motion. The probe 110 pose at the i-th acquisition point as a transformation from the robot base frame {F_{base}} to the PA probe frame {F_{PA}}, denoted as Tᵢ ∈S£(3), is also recorded. A number of N probe poses and N corresponding 2D PA images will be obtained upon completion of the scanning. A 3D PA volume can be generated by transforming all PA images to a common coordinate system (i.e., the robot base frame) utilizing the recorded transformation matrices. For each pixel P^{PA} in the i-th collected PA image under {F^{PA}}, its voxel coordinate (discretized) under the {F_{base}}, P^{base} is calculated by the equation below (the coordinates are homogeneous):

└Pᵢ^{base}┘=TᵢPᵢ^{PA}

The procedure to generate the PA intensity volume is also applied to obtain volumetric data containing functional information by simply replacing the PA intensity with the functional measurement. The returned PA signal 132 is processed to identify oxygen saturation at each imaging plane scan. FIG. 4A shows a Porcine liver sample used for ex vivo study. FIG. 4B shows a depth encoded tissue surface map and FIG. 4C shows an oxygen saturation map of the sample. A complete scan of the specimen 150 area is computed by aggregating or stitching the scan data from individual imaging planes 156 iterated across the specimen 150 according to the path 304.

FIG. 5 shows a laparoscopic use case based on the configuration of FIGS. 2A-2C. In the robotic actuated scan approach of FIGS. 3 and 4A-4C, the sensor 120 remains generally on the same plane above the specimen and retains the same downward, perpendicular pose relative to the plane. This provides a series of parallel imaging planes representing “slices” through the specimen. In a laparoscopic (or endoscopic or other keyhole, minimally invasive approach) the sensor often travels a non-linear path as it tracks anatomical features. Thus, the imaging planes 154 may not be parallel and may even intersect when the sensor follows a convex path or region.

Referring to FIGS. 1, 2A-2C and 5, a laparoscopic probe 510 has a proximal end 103 for surgical manipulation and a distal end 105 for insertion into the surgical region, typically via a laparoscopic incision. The distal end 105 has the photoacoustic sensor 120, the illumination sources 130 and adds one or more localization markers 111 at a relative location from the photoacoustic sensor 120 for coalescing the relative position of the localization marker to the imaging planes 154-1 . . . 154-5 and corresponding coordinate frame 155. It should be apparent that the illumination source(s) 130 are aligned with the photoacoustic sensor 120, which is responsive to the photoacoustic signals 122 induced from the imaging plane 154 from pulsed irradiation by the illumination source 130. The illumination source 130 is therefore adjacent the photoacoustic sensor 120 at the distal end 105 and the illumination source 130 and photoacoustic sensor 120 are fixed in a pose aligned with the imaging plane 154.

As the surgical region is internal to the specimen 150, a second laparoscopic probe introduces a camera 520 for gathering a visual representation of the imaged region in conjunction with the localization markers 111 affixed to the photoacoustic sensor 120 probe 110, such that the camera gathers a position of the localization marker 111 for determining the relative location in the coordinate frame 155 at the time the photoacoustic sensor 120 receives the photoacoustic signals 122 defining a respective imaging plane 154. The position of the localization marker 111 is thus indicative of the coordinate based position of the respective imaging plane 154.

The processor 180 is further configured to map each of the imaging planes 154 to a position in the coordinate frame 155 by computing a position of the localization marker 111 relative to the imaging plane 154 during reception of the photoacoustic signals of the respective imaging plane. This places each successive imaging plane 154-N in a relative position for reconstructing the full image based on the coordinate plane 154.

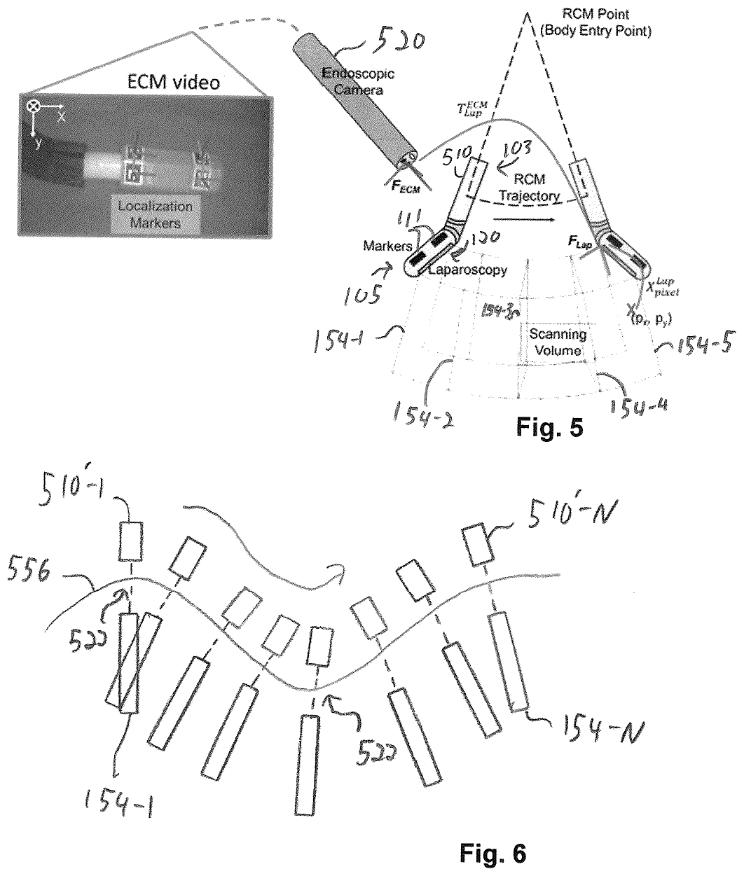

FIG. 6 shows a graphical depiction of the set of scan data defined by a plurality of gathered imaging planes in the approach of FIG. 5. As the laparoscopic probe 510 traverses laparoscopic surface 556, concave/convex surface irregularities 522 occur. As the laparoscopic probe 510 moves tangent to the surface 556, a pose of the transducer 510-1 . . . 510-N(510 generally), changes, along with the corresponding imaging plane which is substantially perpendicular to the tangent path. Accordingly, the resulting set of imaging planes 510 are not parallel but angled and intersecting.

In this manner, the laparoscopic probe 110/510 provides for identifying a pose corresponding to each of the imaging planes, wherein a first imaging plane of the plurality of the imaging planes has a non-parallel orientation to a second imaging plane of the plurality of imaging planes to generate a volumetric rendering.

In order to project functional information that reveals the vasculature under the tissue surface into the endoscopic view for surgical guidance, a sweeping motion of the PA probe is programmed to acquire volumetric PA data that covers a large area in the surgical scene. Cross-sectional PA 3D volume using the tracked probe poses at each imaging location. As illustrated in FIG. 5, the PA probe 510 sweeps on a fan-shape trajectory, with a center located at the probe's 510 body entry point. In the meantime, a total number of m frames PA images were discretely sampled at fixed angular intervals. We assume the centroids of the markers 111 are co-planar, and the rigid body transformation between the arbitrary marker and F_{Lap} is known from the PA probe's Computer-Aided Design (CAD) modeling. Therefore, at the i-th PA image acquisition point, the transformation T_{Lap}^{ECM}₁∈SE(3) from F_{ECM} to F_{Lap} can be derived when at least one marker is captured in the endoscopic view. Multiple markers were able to be detected under most circumstances, where T_{Lap}^{ECM}ₗ was then an average of all possible T_{Lap}^{ECM}_{l,k} derived by the k-th detected marker. Additionally, a temporal averaging filter with a window size of ten timestamps was applied at each acquisition point to reduce the chance of getting an outlier T_{Lap}^{ECM}₁. Lastly, an arc was fitted to the previous i-1 probe poses (i.e., T_{Lap 1}^{ECM}, T_{Lap2}^{ECM}, . . . , T_{Lap i-1}^{ECM}) in the RCM plane to obtain a smoothed historical probe trajectory. The z-axis of F_{Lap} on the smoothed trajectory was resampled to be the tangential direction at each fitted acquisition point, whereas the x-axis was resampled to be the radial direction. Next, the PA volume was generated by transforming all m PA images into a common coordinate, F_{ECM}. For each pixel p^{PA} ∈ [symbol] ² in the i-th PA image, its spatial location P^{Lap} ∈ [symbol] ³ under F_{Lap} can be determined since the physical size of the pixel was known. P^{Lap} was then transformed into F^{ECM} using:

$${p}^{ECM} = {{T}_{Lap}^{{ECM}^{*}}}_{I} \cdot {p}^{Lap}$$

where P^{ECM} is the transformed pixel position. All pixel positions were augmented to be homogeneous with the transformation matrices before multiplication. The volume data, denoted as V, is stored in the form of a 3D matrix by discretizing the transformed pixel positions under F_{ECM}. To better evaluate the accuracy of the volumetric data, later on in section C, we will use maximum intensity projection (MIP) which keeps the highest-intensity pixels in the camera depth direction of V, to visualize V, hence compressing the 3D volume into a 2D image. Note that each pixel in the acquired data is represented in Cartesian coordinates with respect to the imaging probe. The localization marker 111 used for registration was also detected by the camera 520 in Cartesian coordinates. This ensures that all the subsequent processing steps, including the ric imaging and the MIP by taking the maximum intensities along each column, are performed in the Cartesian coordinate system.

While the system and methods defined herein have been particularly shown and described with references to embodiments thereof, it will be understood by those skilled in the art that various changes in form and details may be made therein without departing from the scope of the invention encompassed by the appended claims.

What is claimed is:

1. A medical imaging device, comprising:
a photoacoustic sensor responsive to photoacoustic signals generated based on an imaging plane;
a memory for storing:
a coordinate frame defined over an imaged region; and
an image sequence including a plurality of imaging planes, each imaging plane of the plurality of imaging planes defined by photoacoustic signals representing a two dimensional scan of the imaged region received by the photoacoustic sensor;
a processor for:
coalescing each of the imaging planes of the imaged region with a relative location in the coordinate frame; and
rendering a representation of the imaged region by reconstructing the representation based on the plurality of imaging planes and the corresponding relative location.

2. The device of claim 1 further comprising:
a scanned representation of the imaged region, the coordinate frame derived from the scanned image and indicative of, for each of the imaging planes, a coordinate-based position in the coordinate frame.

3. The device of claim 2 further comprising
a camera, the camera gathering a visual representation of the imaged region; and
a localization marker affixed to the photoacoustic sensor, the camera gathering a position of the localization marker for determining the relative location in the coordinate frame at the time the photoacoustic sensor receives the photoacoustic signals defining a respective imaging plane, the position of the localization marker indicative of the coordinate based position of the respective imaging plane.

4. The device of claim 3 wherein the processor is further configured to map each of the imaging planes to a position in the coordinate frame by computing a position of the localization marker relative to the imaging plane during reception of the photoacoustic signals of the respective imaging plane.

5. The device of claim 2 further comprising:
a robotic actuator, the photoacoustic sensor attached to the robotic actuator, the robotic actuator adapted to:
traverse a predetermined path relative to the imaged region; and
compute the coordinate based position of the respective imaging plane based on a position on the predetermined path when the photoacoustic signals corresponding to the imaging plane are received by the photoacoustic sensor.

6. The device of claim 5 wherein the robotic actuator follows a series of parallel swaths along a plane adjacent to the imaged region.

7. The device of claim 1 further comprising a laparoscopic probe, the laparoscopic probe having a proximal end for surgical manipulation and a distal end, the distal end having:
the photoacoustic sensor; and
the localization marker at a relative location from the photoacoustic sensor for coalescing the relative position of the localization marker to the imaging plane.

8. The device of claim 1, further comprising:
an illumination source, the illumination source aligned with the photoacoustic sensor, the photoacoustic sensor responsive to photoacoustic signals induced from the imaging plane from pulsed irradiation by the illumination source.

9. The device of claim 7, further comprising an illumination source adjacent the photoacoustic sensor at the distal end, the illumination source and photoacoustic sensor fixed in a pose aligned with the imaging plane.

10. The device of claim 3 wherein the processor is configured for identifying a pose corresponding to each of the imaging planes, wherein a first imaging plane of the plurality of the imaging planes has a non-parallel orientation to a second imaging plane of the plurality of imaging planes.

11. A method for photoacoustic imaging, comprising:
receiving photoacoustic signals generated based on a sequence of imaging planes depicting an imaged region to form an image sequence including a plurality of imaging planes, each imaging plane of the plurality of imaging planes defined by photoacoustic signals representing a two dimensional scan of the imaged region received by a photoacoustic sensor
defining a coordinate frame defined over the imaged region; and
coalescing each of the imaging planes of the imaged region with a relative location in the coordinate frame; and
rendering a representation of the imaged region by reconstructing the representation based on the plurality of imaging planes and the corresponding relative location.
